(19) United States
(12) Patent Application Publication
(10) Pub. No.: US 2026/0370800 A1
(43) Pub. Date: Nov. 19, 2026

(54) TARGET CONFIGURED TO TEST A MICROSCOPE SYSTEM

(71) Applicant: ALCON INC., Fribourg (CH)

(72) Inventors: Friederike Rubin-Schwarz, Nuernberg (DE); Annelene Konz, Hollfeld (DE); Wolfgang Raab, Nuernberg (DE)

(21) Appl. No.: 19/674,342

(22) Filed: May 12, 2026

Related U.S. Application Data
(60) Provisional application No. 63/806,045, filed on May 15, 2025.

(51) Int. Cl. H04N 17/00; A61B 3/13; G06T 7/521; G06T 7/80; H04N 17/02

(52) U.S. Cl. CPC H04N 17/002; A61B 3/13; G06T 7/521; G06T 7/85; H04N 17/02; G06T 2207/10012; G06T 2207/10024; G06T 2207/10028; G06T 2207/10056; G06T 2207/30041

(57) ABSTRACT

In at least one embodiment, a target is configured to test an imaging system. The imaging system includes cameras, such as a first camera and a second camera. The target includes a base and a wall. The base has an upper base surface. Graphics are located on the upper base surface. The graphics are designed to facilitate testing of the imaging system and include a first graphic and a second graphic. The wall is disposed outwardly from the base. The wall has a top disposed on a side opposite of the base. When the first camera and the second camera are aligned, the wall allows the first camera to image the first graphic, allows the second camera to image the second graphic, blocks the first camera from imaging the second graphic, and blocks the second camera from imaging the first graphic.

### FIELD

Embodiments of the present disclosure relate to a target configured to test an imaging system.

### BACKGROUND

A medical system such as an ophthalmic system may include a microscope system used to perform a medical procedure. The microscope system may have a digital imaging system with one or more cameras. A camera may have settings, such as alignment, that should be checked prior to performing the procedure. Other settings, such as the resolution of the camera, may also be checked.

### SUMMARY

In at least one embodiment, a target is configured to test an imaging system. The imaging system includes cameras, such as a first camera and a second camera. The target includes a base and a wall. The base has an upper base surface. Graphics are located on the upper base surface. The graphics are designed to facilitate testing of the imaging system and include a first graphic and a second graphic. The wall is disposed outwardly from the base. The wall has a top disposed on a side opposite of the base. When the first camera and the second camera are aligned, the wall allows the first camera to image the first graphic, allows the second camera to image the second graphic, blocks the first camera from imaging the second graphic, and blocks the second camera from imaging the first graphic.

One or more embodiments may include none, one, two or more, or all of the following:
The top has a top midplane, and the wall has a wall midplane. The top midplane is substantially orthogonal to the wall midplane.
The wall has a wall midplane, and the base has a base midplane. The wall midplane is substantially orthogonal to the base midplane.
The top has an upper top surface. One or more of the graphics is located on the upper top surface.
The graphics include a color test graphic that facilitates testing of a color response of at least one of the cameras.
The graphics include a resolution test graphic that facilitates testing of a resolution of at least one of the cameras.
The graphics include a field-of-view test graphic that facilitates testing of a field-of-view of at least one of the cameras.
The graphics include a pilot test graphic that indicates an aligned location of a pilot laser spot.
The graphics include a distance test graphic that indicates at least one aligned location of a distance laser spot.
The imaging system has an optical axis. The first camera is a first off-axis camera relative to the optical axis, and the second camera is a second off-axis camera relative to the optical axis.

The top has a top midplane and at least one overhang that extends along the top midplane away from the wall. The overhang may have a top window. The top window may be an indentation defined by an edge of the overhang or may be a transparent portion of the overhang. The top may have multiple overhangs, such as a first overhang and a second overhang, where the first overhang has a first top window that allows the first camera to image the first graphic, and the second overhang has a second top window that allows the second camera to image the second graphic. The overhang may have a top window with a window graphic, where the top window allows the first camera to image the first graphic and the window graphic, and a spatial relationship between the first graphic and the window graphic indicates an alignment of the first camera.

The wall has a rectangular cuboid shape. An upper surface of the wall forms the top of the wall. The wall has a line of sight channel that allows the first camera to image the first graphic. The line of sight channel may be an indentation of the wall or may be a transparent portion of the wall.

In at least one embodiment, an ophthalmic system includes an imaging system and a computer system. The imaging system includes a first camera and a second camera. The first camera generates a first image of a target. The second camera generates a second image of the target. The target has a base and a wall, where the wall is disposed outwardly from the base. The base has a first graphic and a second graphic designed to facilitate testing of the imaging system. When the first camera and the second camera are aligned, the wall allows the first camera to image the first graphic, allows the second camera to image the second graphic, blocks the first camera from imaging the second graphic, and blocks the second camera from imaging the first graphic. The computer system establishes that the first image shows the first graphic but not the second graphic, establishes that the second image shows the second graphic but not the first graphic, and determines that the first camera and the second camera are aligned.

One or more embodiments may include none, one, two or more, or all of the following:
The ophthalmic system further includes a laser system that directs a pilot beam towards the target to yield a pilot beam spot on the target. The pilot beam indicates a direction of a laser beam provided by the laser system. The target has a pilot test graphic indicating an alignment of the pilot beam. The imaging system generates a pilot beam image of the pilot beam spot on the target. The computer system determines the alignment of the pilot beam relative to the target according to a location of the pilot beam spot relative to the pilot test graphic.
The ophthalmic system further includes an alignment light system that directs off-axis light beams towards the target to yield off-axis light spots on the target. The off-axis light beams indicating an aligned distance between the ophthalmic system and the target. The target has a distance test graphic corresponding to the off-axis light beams. The imaging system generates a distance image of the plurality of off-axis light spots on the target. The computer system determines whether a distance between the ophthalmic system and the target corresponds to the aligned distance according to a location of the off-axis light spots relative to the distance test graphic.
The ophthalmic system further includes a platform configured to support the target. The computer system determine a misalignment of the target relative to the ophthalmic system and instruct the platform to move to compensate for the misalignment.

### BRIEF DESCRIPTION OF THE DRAWINGS

FIGS. 1A-1D illustrate an example of a system with a microscope system that includes an imaging system that may be tested using a target, according to at least one embodiment described in the present disclosure;

FIGS. 2A-2G illustrate an example of a target, according to at least one embodiment described in the present disclosure;

FIGS. 3A-3D illustrate an example of a target with one or more top windows, according to at least one embodiment described in the present disclosure;

FIGS. 4A and 4B illustrate an example of a target with one or more line of sight (LOS) channels, according to at least one embodiment described in the present disclosure; and

FIG. 5 illustrates an example of a method of using a target to test an imaging system, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

A medical system such as an ophthalmic system may include a microscope system with settings that may need to be tested, e.g., evaluated and/or verified. The present disclosure relates to a target that may be used to test the settings of a microscope system. For example, a microscope system may include a stereoscopic system with a left camera and a right camera, and the target may have a left graphic and a right graphic that can be used to test the left camera and the right camera, respectively. The target may be configured to allow the left camera to image the left graphic, but not the right graphic if properly aligned. Similarly, the target may be configured to allow the right camera to image the right graphic, but not the left graphic if properly aligned. The target may also include other graphics designed to facilitate testing of, e.g., a field-of-view, a color response, and/or a resolution of the microscope system. As another example, the ophthalmic system may include a computer system that can analyze an image taken by a camera of a microscope system, e.g., an image taken by a left camera of a stereoscopic system. The computer system may determine the alignment of the left camera according to whether a left graphic, but not a right graphic, appears in the image.

Certain embodiments of the present disclosure may provide improvements over known iterations of targets. For example, a target may be used to test a left camera and a right camera of a stereoscopic system. As another example, the target may be used to test different settings of a microscope system, e.g., a field-of-view, a color response, and/or a resolution of the microscope system. As another example, a computer system may analyze an image of the target generated by a camera to test, e.g., the alignment of the camera.

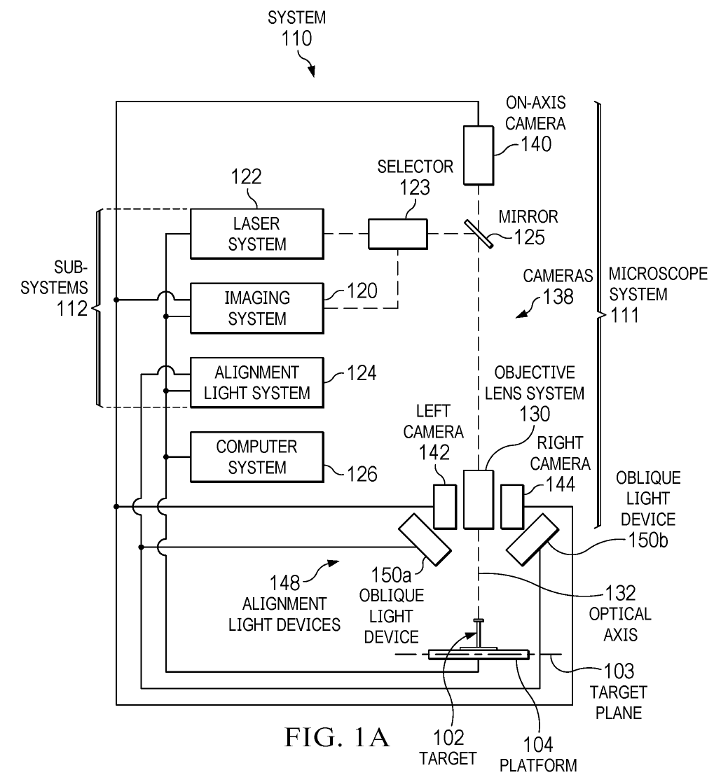

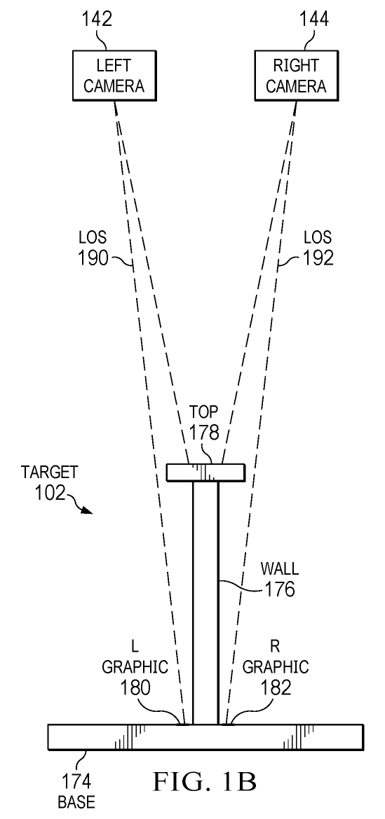

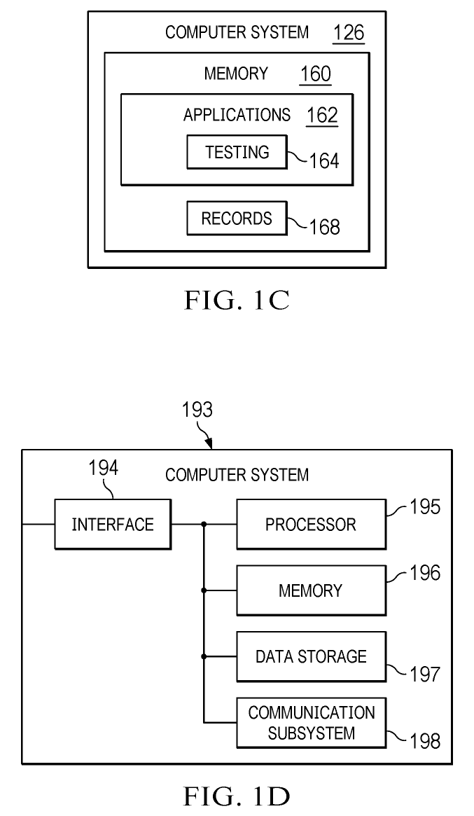

FIGS. 1A-1D illustrate an example of a system 110 with a microscope system 111 that includes an imaging system 120 that may be tested using a target 102, according to at least one embodiment described in the present disclosure. FIG. 1A shows a block diagram view of the system 110. FIG. 1B shows an example of the target 102. FIG. 1C shows an example of a computer system 126 that may be used with the system 110. FIG. 1D shows another example of a computer system 193 that may be used with the system 110.

In one or more embodiments, the system 110 may be an ophthalmic system designed to perform a procedure (e.g., a surgical and/or diagnostic procedure) on an eye. The target 102 may be used to check settings of the system 110 (e.g., alignment) in order to perform the procedure on the eye. The system 110 may be checked during, e.g., manufacturing and/or clinical use.

As an overview of the example, the system 110 includes the microscope system 111, the subsystems 112, and/or a computer system 126, which may be coupled (e.g., electronically, mechanically, optically, communicatively, and/or otherwise coupled) as shown. The microscope system 111 includes the imaging system 120, a selector 123, a mirror 125, and/or an objective lens system 130 (with an optical axis 132). The imaging system 120 includes one or more cameras 138, e.g., an on-axis camera 140, a left camera (“L camera”) 142, and/or a right camera (“R camera”) 144, which may be coupled as shown. The left camera 142 has a line of sight (LOS) 190, and the right camera 144 has a line of sight (LOS) 192. The subsystems 112 include the imaging system 120, a laser system 122, and/or an alignment light system 124, which may be coupled as shown. The alignment light system 124 includes alignment light devices 148, e.g., one or more oblique light devices 150 (150a and/or 150b), which may be coupled as shown. The computer system 126 includes a memory 160, which stores software applications 162 (e.g., a testing application 164) and records 168. A platform 104 supports the target 102 at a target plane 103. The target 102 has a base 174 and a wall 176 (which has a top 178). The base 172 has a left “L” graphic 180 and a right “R” graphic 182.

In an example of operation, the target 102 may be used to test the left camera 142 and the right camera 144 of the microscope system 111. In the example, when the left camera 142 is aligned, the wall 176 of the target 102 allows the line of sight 190 of the left camera 142 to reach the left graphic 180, but blocks the line of sight 190 from reaching the right graphic 182. That is, the wall 176 allows the left camera 142 to image the left graphic 180, but not the right graphic 182. Similarly, when the right camera 144 is aligned, the wall 176 allows the line of sight 192 of the right camera 144 to reach the right graphic 182, but blocks the line of sight 190 from reaching the left graphic 180. That is, the wall 176 allows the right camera 144 to image the right graphic 182, but not the left graphic 180. The target 102 may also include graphics designed to facilitate testing of, e.g., a field-of-view, a color response, and/or a resolution of one or more cameras 138. Other graphics may be used to align the target 102 with the system 110. For example, one or more graphics may be used to align the target 102 in a direction along the optical axis 132 and/or in a direction orthogonal to the optical axis 132.

In one or more embodiments, allowing a camera to image a graphic may mean allowing the camera to image most of the graphic, e.g., at least 50 to 100 percent of the graphic, such as at least 50 to 60 percent, 60 to 70 percent, 70 to 80 percent, 80 to 90 percent, and/or 90 to 100 percent of the graphic. In one or more embodiments, blocking a camera from imaging a graphic may mean blocking the camera from imaging most of the graphic, as described previously.

Turning to the components of the system 110, the system 110 includes the laser system 122, which directs a laser beam towards the target 102. The laser system 122 may include a laser source that generates the laser beam and a scanner that guides the laser beam to points of the target 102. The laser beam may have any suitable pulse duration, such as in the order of nanoseconds, picoseconds, femtoseconds, or attoseconds. The laser beam may have any suitable wavelength, such as in the range of 150 nanometers (nm) to 20 micrometers (µm). Examples of ranges include the ultraviolet (e.g., in the range of 180 to 400 nm, such as 190 to 195 nm and/or 345 to 355 nm), visible, or infrared (including near-infrared) wavelength (e.g., in the range of 1000 to 1250 and/or 1250 to 1500 nm). In one or more embodiments, the laser system 122 directs a pilot laser beam (or “pilot beam”) towards the target 102, where the pilot beam indicates a direction of the laser beam.

The imaging system 120 generates of images of the target 102. The imaging system 120 may include one or more cameras 138. A camera 138 may be any suitable system that detects light reflected from the target 102 to generate an image of the target 102. Examples of such systems include a digital sensor, a digital camera, and/or an optical coherence tomography (OCT) system. A camera may detect any suitable wavelength range of light, e.g., visible light, ultraviolet (UV) light, infrared light, and/or near infrared light. In the example, the cameras 138 include, e.g., the on-axis camera 140, the left camera 142, and/or the right camera 144. The on-axis camera 140 may have an optical axis that aligns with the optical axis 132 of the objective lens system 130. The left camera 142 and/or the right camera 144 may be off-axis cameras. The left camera 142 has an optical axis that is at an offset angle relative to the optical axis 132 of the objective lens system 130. The right camera 144 has an optical axis that is also at an offset angle relative to the optical axis 132. The optical axes may be offset in opposite directions to yield a stereoscopic image. An offset angle may have any suitable value, e.g., 1 to 45 degrees, such as 1 to 15, 15 to 30, and/or 30 to 45 degrees.

In other examples, the imaging system 120 may include an OCT system that generates an imaging beam, which travels via the selector 123, the mirror 125, and/or the objective lens system 130 to the target 102. The target 102 reflects the imaging beam, which travels back to the imaging system 120. The imaging system 120 analyzes the reflected imaging beam to generate an OCT image of the target 102.

The alignment light system 124 includes one or more alignment light devices 148. An alignment light device 148 directs light towards the target 102 to yield a light spot on the target 102, which may guide placement of the target 102 at the proper position (e.g., location and/or orientation relative to the target plane 103 and/or the optical axis 132) to test the imaging system 120. Any suitable wavelength of light may be used, e.g., visible light, ultraviolet (UV) light, infrared light, and/or near-infrared light. In the example, the alignment light devices 148 includes, e.g., the oblique light devices 150 (150a and/or 150b). An oblique light device 150 (150a or 150b) directs light at an offset angle relative to the optical axis 132 of the objective lens system 130. In an example, each oblique light devices 150 (150a or 150b) directs an off-axis light beam towards the target plane 103. The light spots created by the light beams may indicate a distance relative to the target plane. For example, the light spots may intersect, e.g., appear as one light spot, at the target plane 103.

The selector 123 selects the laser beam from the laser system 122 and/or the imaging beam from the imaging system 120 to direct towards the mirror 125, which directs the selected beam towards the objective lens system 130. The objective lens system 130 includes an objective lens that directs light, e.g., an alignment light beam, a light beam, and/or a laser beam, towards the target 102. The objective lens system 130 may also direct light from the target 102 towards, e.g., one or more of the cameras 138. The objective lens system 130 has an optical axis 132, which may be an optical axis of the objective lens. In one or more embodiments, the optical axis 132 may be used to align the target 102.

The computer system 126 performs operations for the system 110. The computer system 126 may send instructions to components of the system 110, e.g., the laser system 122, the imaging system 120, and/or the alignment light system 124. The computer system 126 may utilize applications 162 (e.g., the testing application 164) and records 168 to perform the operations. The applications 162 may also include image processing software that can be used to analyze an image of the target 102 generated by a camera to test the camera.

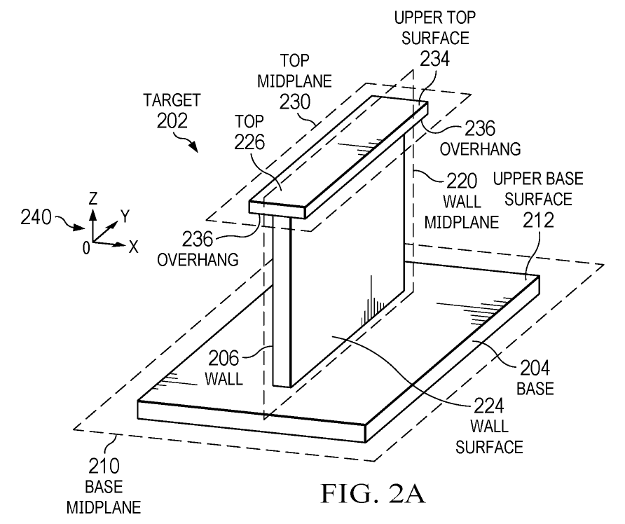

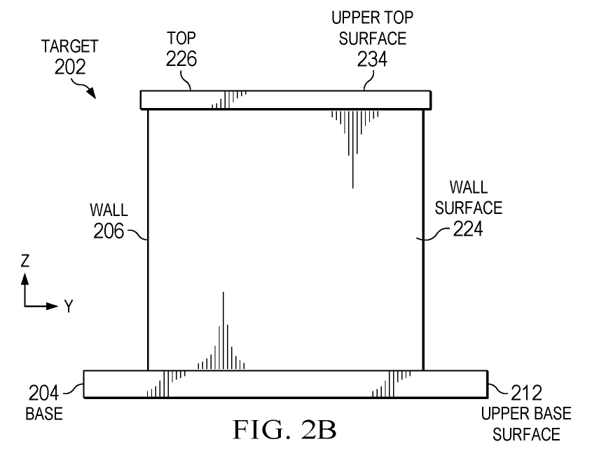

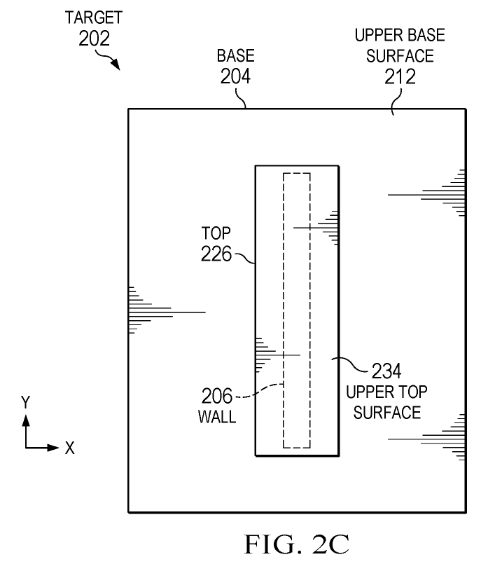

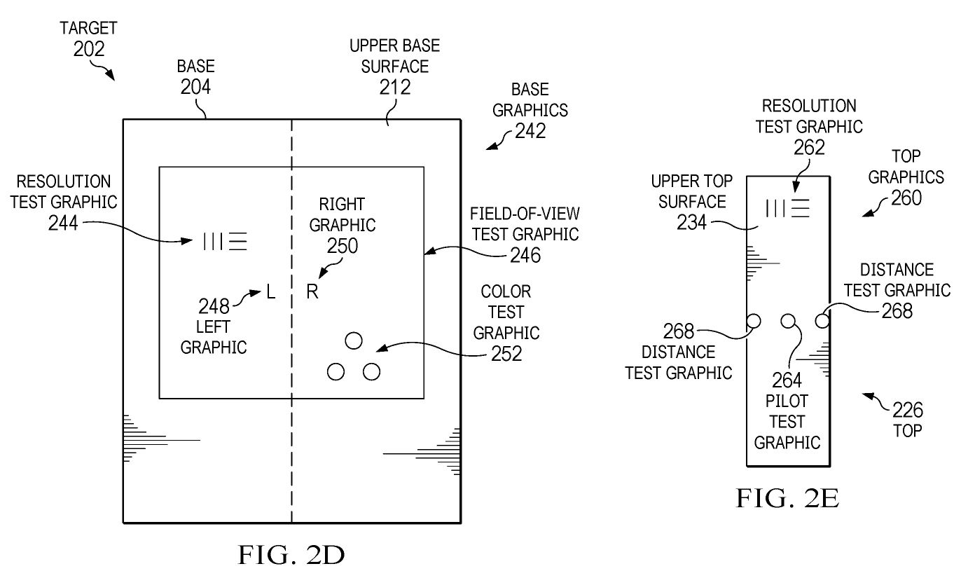

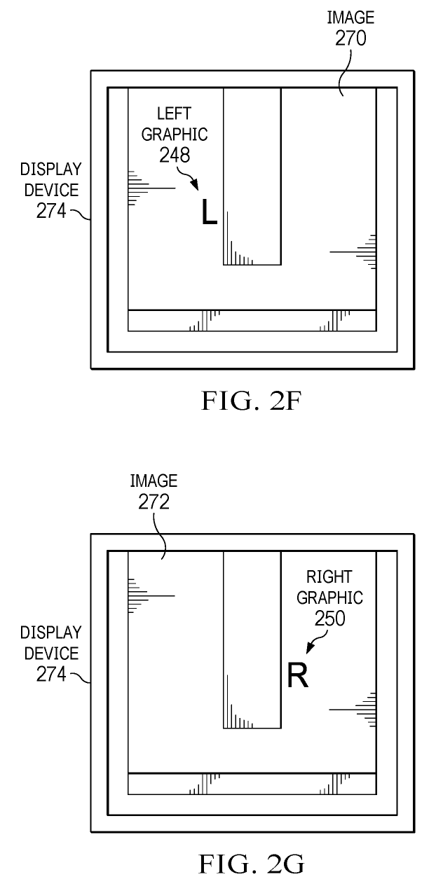

In one or more embodiments, the computer system 126 may use the testing application 164 to instruct the imaging system 120 to generate images of the target 102. The testing application 164 may then be used to analyze the images to check the imaging system 120. For example, the left camera 142 generates a left image of the target 102, and the right camera 144 generates a right image of the target 102. The computer system 126 establishes that the left image shows the left graphic 180 but not the right graphic 182 and/or the right image shows the left graphic but not the right graphic. The computer system 126 then determines that the first camera and/or the second camera are aligned, as described in more detail herein, e.g., with reference to FIGS. 2A-2G.

In one or more embodiments, the computer system 126 may instruct a subsystem 112 to direct a light beam towards the target 102, instruct the imaging system 120 to generate an image of the target 102, and then analyze the image to determine if the target 102 and/or the system 110 is properly aligned. For example, the laser system 122 directs a pilot beam towards the target 102, where the pilot beam indicates a direction of a laser beam provided by the laser system 122. The target 102 has a pilot test graphic designed to facilitate testing of an alignment of the target 102 relative to the laser system 122 in, e.g., a direction orthogonal to the optical axis 132. The pilot beam yields a pilot beam spot on the target 102. The on-axis camera 140 generates a pilot beam image of the pilot beam spot on the target 102. The computer system 126 analyzes the location of the pilot beam spot relative to the pilot test graphic to determine, e.g., the alignment of the target 102 relative to the laser system 122 and/or the alignment of the pilot beam relative to the target 102, as described herein, e.g., with reference to FIGS. 2D and 2E.

As another example, the alignment light system 124 directs off-axis light beams towards the target 102, where the light beams indicate a distance between the system 110 and the target 102 in the direction parallel to the optical axis 132. The off-axis light beams yield off-axis light spots on the target 102. The target 102 has a distance test graphic corresponding to the off-axis light beams. The imaging system 120 generates a distance image of the off-axis light spots and the distance test graphic. The computer system 126 determines a distance between the system 110 and the target 102 according to a location of the off-axis light spots relative to the distance test graphic, as described herein, e.g., with reference to FIGS. 2D and 2E.

In one or more embodiments, the target 102 may be supported by (e.g., be placed onto) the platform 104 in order to test the system 110. The computer system 126 may detect that the target 102 is not aligned with the system 110 and may move the platform 104 to align the target 102 with the system 110. For example, the computer system 126 may determine whether the target 102 is aligned in one or more directions parallel to and/or orthogonal to the optical axis 132. If the target is not aligned in a direction of the one or more directions, the computer system 126 may move the platform 104 to compensate for the misalignment.

In some embodiments, the platform 104 may include a surgical headrest of a bed or other patient support that may or may not be movable under computer control (e.g., the computer system 126 and/or 193). In these and other embodiments, the base 174 may sit upon the headrest. Additionally or alternatively, such a headrest may include a slot or opening into which the wall 176 of the target 102 may be inserted such that part of the headrest forms the base 174. For example, the left graphic 180 and/or the right graphic 182 may be stitched or printed onto the headrest on either side of the slot into which the wall 176 is insertable. In these and other embodiments, the headrest may have its position adjusted (whether automatically under computer control or manually by a healthcare professional) such that the base 174 is at a height/depth relative to the objective lens system 130 of a typical surgery involving the laser system 122.

In one or more embodiments, the computer system 126 may send a notification if a test result is not acceptable. In one or more embodiments, the computer system 126 may save information in the records 168 that document the results of the tests with the target 102. For example, the computer system 126 may save one or more images of the target 102. Saving images taken over time with the same target 102 may allow for more consistent documentation of the system 110. Additionally or alternatively, if the platform 104 is movable, the platform 104 may be moved until the target 102 is in alignment such that the test result is acceptable. For example, a computer system may automatically move the platform 104, followed by re-performing the test, and iteratively doing so until the test result is acceptable. As another example, a healthcare professional may move the platform 104 manually, trigger a repeat of the test, observe the results, and if not acceptable, further move the platform 104. Such a process may be repeated with repeated manual movement of the platform 104 and triggering of the test until the test results are acceptable.

FIG. 1D illustrates an example computer system 193, according to at least one embodiment described in the present disclosure. The computer system 193 may include an interface 194, a processor 195, a memory 196, a data storage 197, and/or a communication subsystem 198, any or all of which may be communicatively coupled. Any or all of the computer system 193 may be implemented as computer hardware and/or software. Any or all of the systems described herein may be implemented as a computer system consistent with the computer system 193.

In the example, the interface 194 may receive input to the computer system 193 and/or send output from the computer system 193, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computer system 193. Examples of user interfaces include displays, Graphical User Interfaces (GUIs), touchscreens, foot pedals, keyboards, computer mouses (or mice), gesture sensors, microphones, and speakers.

Generally, the processor 195 may include any suitable special-purpose or general-purpose computer, computing entity, or processing device including various computer hardware or software modules and may be configured to execute instructions stored on any applicable computer-readable storage media. For example, the processor 195 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor, the processor 195 may include any number of processors distributed across any number of network or physical locations that are configured to perform individually or collectively any number of operations described in the present disclosure.

The processor 195 may perform any suitable operations. In some embodiments, the processor 195 may interpret and/or execute program instructions and/or process data stored in the memory 196, the data storage 197, or the memory 196 and the data storage 197. In some embodiments, the processor 195 may fetch program instructions from the data storage 197 and load the program instructions into the memory 196. After the program instructions are loaded into the memory 196, the processor 195 may execute the program instructions, such as instructions to perform any of the methods disclosed herein, respectively.

The memory 196 and the data storage 197 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 195.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 195 to perform a certain operation or group of operations.

The communication subsystem 198 may include any component, device, system, or combination thereof that is configured to transmit, receive, and/or otherwise exchange information over a network in order to communicate with any suitable entity, such as with other devices at other locations or at the same location or even within the same system. The communication subsystem 198 may provide for communication among the devices described in the present disclosure, communication networks, computing devices, and other systems. For example, the communication subsystem 198 may allow the system 193 to communicate with other systems, such as other computing devices and/or networks. In some embodiments, the communication subsystem 198 may include a modem, a network card (wireless or wired), an optical communication device, an infrared communication device, a wireless communication device (such as an antenna), and/or chipset. Examples of communication subsystem 198 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the system 193 without departing from the scope of the present disclosure. For example, the system 193 may include more or fewer components than those explicitly illustrated and described.

FIGS. 2A-2G illustrate an example of a target 202, according to at least one embodiment described in the present disclosure. FIG. 2A shows a perspectival view of the target 202, FIG. 2B shows a side view of the target 202, and FIG. 2C shows a top view of the target 202. FIG. 2D shows the base 204 of the target 202, and FIG. 2E shows the top 226 of the target 202. FIG. 2F shows an image 270 of the target 202 from a left camera displayed on a display device 274, and FIG. 2G shows an image 272 of the target 202 from a right camera displayed on the display device 274.

As an overview of the example, the target 202 includes a base 204 and a wall 206, which may be coupled as shown. The base 204 has a base midplane 210 and an upper base surface 212. The wall 206 has a wall midplane 220, a wall surface 224, and a top 226, which may be arranged as shown. The top 226 has a top midplane 230 and an upper top surface 234 and may have one or more overhangs 236. The base 204 has base graphics 242, e.g., a resolution test graphic 244, a field-of-view (FOV) test graphic 246, a left graphic 248, a right graphic 250, and/or a color test graphic 252. The top 226 has top graphics 260, e.g., a resolution test graphic 262, a pilot test graphic 264, and/or a distance test graphic 268.

For ease of explanation, a coordinate system 240 may be used. An origin O of the coordinate system 240 may be located at a midpoint of the base midplane 210. A y-axis may be located where the wall midplane 220 intersects with the base midplane 210. An x-axis may be located on the base midplane 210 and orthogonal to the y-axis. A z-axis may be located on the wall midplane 220 and orthogonal to the y-axis. Of course, any suitable coordinate system may be used.

Turning to a more detailed description of the example target 202, FIG. 2A shows the perspectival view of the target 202, FIG. 2B shows the side view of the target 202, and FIG. 2C shows the top view of the target 202. In the example, the target 202 may be made of any suitable material, e.g., a rigid material such as plastic, metal, wood, glass, ceramic, and/or other rigid material. In one or more embodiments, a camera may be designed to detect a specific wavelength range of light as described herein, e.g., with reference to FIG. 1A. The target 202 may include material that can reflect and/or emit light in the specific wavelength range.

In the example, the target 202 includes the base 204 and the wall 206. The base 204 and the wall 206 may each have any suitable size and/or shape, and the base 204 and the wall 206 may have any suitable arrangement relative to each other. In at least one embodiment, the base 204 and the wall 206 may have a size, shape, and/or arrangement that allows and/or blocks one or more lines of sight of one or more cameras as described with reference to FIG. 1B. For example, the wall midplane 220 may be substantially orthogonal to the base midplane 210.

In the example, the base 204 has a substantially rectangular, planar shape. The base midplane 210 may be substantially parallel to the upper base surface 212. The base 204 may have any suitable length in the y-direction, e.g., 1 to 10 centimeters (cm), such as 1 to 2 cm, 2 to 4 cm, 4 to 6 cm, and/or 6 to 10 cm. The base 204 may have any suitable width in the x-direction, e.g., 1 to 10 centimeters (cm), such as 1 to 2 cm, 2 to 4 cm, 4 to 6 cm, and/or 6 to 10 cm. The base 204 may have any suitable thickness in the z-direction, e.g., 1 to 10 millimeters (mm), such as 1 to 2 mm, 2 to 4 mm, 4 to 6 mm, and/or 6 to 10 mm.

In the example, the wall 206 has a substantially rectangular, planar shape. The wall midplane 220 may be substantially parallel to the wall surface 224, and the top midplane 230 may be substantially parallel to the upper top surface 234. The wall midplane 220 may be substantially orthogonal to the top midplane 230. The wall 206 may have any suitable height in the z-direction, e.g., 1 to 10 centimeters (cm), such as 1 to 2 cm, 2 to 4 cm, 4 to 6 cm, and/or 6 to 10 cm. The wall 206 may have any suitable length in the y-direction, e.g., 1 to 10 centimeters (cm), such as 1 to 2 cm, 2 to 4 cm, 4 to 6 cm, and/or 6 to 10 cm. The wall 206 may have any suitable thickness in the x-direction, e.g., 1 to 10 millimeters (mm), such as 1 to 2 mm, 2 to 4 mm, 4 to 6 mm, and/or 6 to 10 mm.

The top 226 may have any suitable length in the y-direction, e.g., 1 to 10 centimeters (cm), such as 1 to 2 cm, 2 to 4 cm, 4 to 6 cm, and/or 6 to 10 cm. The top 226 may have any suitable width in the x-direction, e.g., 1 to 6 centimeters (cm), such as 1 to 2 cm, 2 to 4 cm, and/or 4 to 6 cm. The top 226 may have any suitable thickness in the z-direction, e.g., 1 to 10 millimeters (mm), such as 1 to 2 mm, 2 to 4 mm, 4 to 6 mm, and/or 6 to 10 mm. In the example, the top 226 has the overhangs 236, where each overhang extends the top midplane 230 away from the wall 206.

Turning to a more detailed description of the example graphics, FIG. 2D shows the base 204 of the target 202 with examples of base graphics 242, and FIG. 2E shows the top 226 of the target 202 with examples of top graphics 260. The base graphics 242 and the top graphics 260 may include any suitable graphics. For example, a graphic that is shown as a base graphic 242 in the example may be a top graphic 260 in another example, and vice-versa. A graphic may be placed on the target 202 in any suitable manner. For example, a graphic may be adhered onto the target 202 (e.g., with a glue or other adhesive), printed and/or painted onto the target 202, inlaid into the target (with, e.g., a metal), carved into the target 202, or placed on the target 202 in any other suitable manner. In one or more embodiments, a camera may be configured to detect a specific wavelength of light as described herein, e.g., with reference to FIG. 1A. The graphic may include material that can reflect and/or emit light in the specific wavelength range.

In the example, the base graphics 242 include the resolution test graphic 244, the field-of-view (FOV) test graphic 246, the left graphic 248, the right graphic 250, and/or the color test graphic 252. The resolution test graphic 244 facilitates testing of a resolution of a camera. The resolution test graphic 244 may include a resolution test pattern. In an example, a resolution test pattern includes reference line patterns with specific thicknesses and spacings that may be placed in the object plane of the camera. The largest set of non-distinguishable lines may be identified to determine the resolution of the camera. An example of a resolution test pattern is the 1951 USAF resolution test chart. The field-of-view (FOV) test graphic 246 outlines the field of view of a camera that is aligned with the system. If the field of view imaged by the camera does not have the same size, shape, and/or location as the FOV test graphic 246, the camera might not be aligned.

The left graphic 248 and the right graphic 250 facilitate testing of the alignment of off-axis cameras, such as a right camera and a left camera, as described herein. In the example, when the cameras are aligned, the right camera should be able to generate the image 272 (shown in FIG. 2G) of the right graphic 250, and the left camera should be able to generate the image 270 (shown in FIG. 2F) of the left graphic 248. The right graphic 250 and the left graphic 248 may also serve to check the connections of the right camera and the left camera. If the right camera yields the image 270 of the left graphic 248, and the left camera yields the image 272 of the right graphic 250, the right camera and the left camera may be incorrectly connected to the system.

The color test graphic 252 facilitates testing of a color response of a camera. The color test graphic 252 may include a color test pattern with known colors for the camera to image. The color test pattern may include graphics (e.g., dots, letters, and/or other shapes) of different colors, e.g., red, green, and/or blue, or any other suitable color combination.

The top 226 has top graphics 260, e.g., a resolution test graphic 262, a pilot test graphic 264, and a distance test graphic 268. The resolution test graphic 262 may be as described with reference to resolution test graphic 244.

In certain embodiments, a pilot beam is used to indicate a direction of a laser beam provided by a laser system. The pilot test graphic 264 is designed to indicate where the pilot beam creates a pilot beam spot on the target 202 when the target 202 and the laser system are aligned with each other. For example, if the pilot beam falls within the circle of the pilot test graphic 264, the target 202 and the laser system may be regarded as aligned with each other.

In certain embodiments, off-axis light beams may be used to indicate a distance between the ophthalmic system and the target 202. The distance test graphic 268 is designed to indicate where the off-axis light beams creates off-axis light spots on the target 202 when the target 202 is at a specific distance away from the system, e.g., the base 204 of the target 202 is at the target plane of the system. For example, if the off-axis light spots fall within the circles of the distance test graphic 268, the base 204 may be regarded as at the target plane.

Of course, the target 202 may have any other suitable graphic not necessarily shown in the illustrated example. For example, the target 202 may have a star target graphic that may be used to detect a focus error and/or an astigmatism of a camera. The star target graphic may include any suitable number of bars (e.g., 50 to 200 bars, such as 50 to 100 bars, 100 to 150 bars, and/or 150 to 200 bars) that radiate from a central point. As another example, the target 202 may have a variable frequency target graphic that may be used to evaluate the resolution, field distortion, and/or parfocal stability of a camera. The variable frequency target graphic may include any suitable number of line pairs per millimeter (lp/mm), e.g., 5 to 250 lp/mm, such as 5 to 50 lp/mm, 50 to 100 lp/mm, 100 to 150 lp/mm, and/or 150 to 250 lp/mm.

In some embodiments, one or more of the base graphics 242 and/or the top graphics 260 may be combined. For example, the text of the left graphic 248 and/or the right graphic 250 may be printed in or otherwise comprised of one or more colors. This may permit the left graphic 248 and/or the right graphic 250 to take the place of the color test graphic 252. As another example, multiple instances of the left graphic 248 may be included of varying sizes to test the resolutions of the left and/or right cameras, thereby filling the role of the resolution test graphic 244.

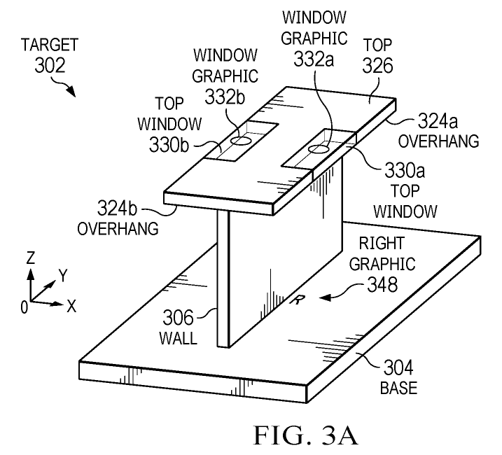

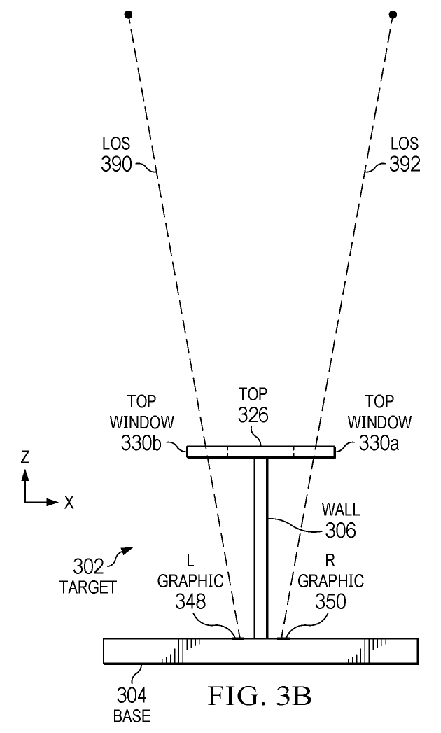

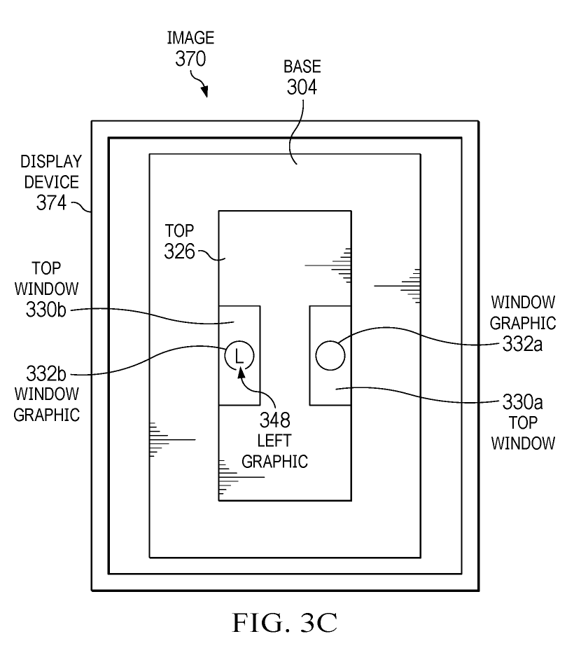

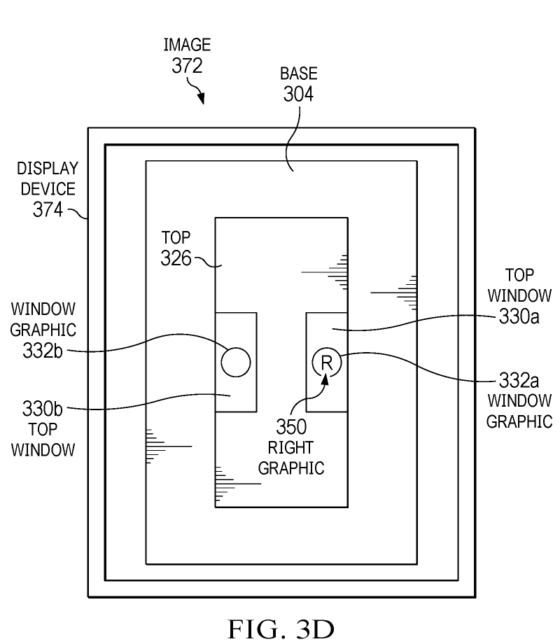

FIGS. 3A-3D illustrate an example of a target 302 with one or more top windows 330 (330a and/or 330b), according to at least one embodiment described in the present disclosure. In the example, the target 302 may be used to test a left camera and a right camera of an imaging system. FIG. 3A shows a perspectival view of the target 302. FIG. 3B shows the target 302 relative to a line of sight (LOS) 390 of the left camera and a line of sight (LOS) 392 of the right camera. FIG. 3C shows a display device 374 displaying an image 370 of the target 302 from the left camera, and FIG. 3G shows the display device 374 displaying an image 372 of the target 302 from the right camera.

As an overview of the example, the target 302 includes a base 304 and/or a wall 306, which may be coupled as shown. The base has a left graphic 348 and a right graphic 350. The wall 306 has a top 326 that has one or more overhangs 324 (324a and/or 324b). In the example, each overhang (324a or 324b) includes a top window 330 (330a or 330b, respectively), which has a window graphic 332 (332a or 332b, respectively). The line of sight 390 of the left camera passes through the top window 330b to the left graphic 348, and the line of sight 392 of the right camera passes through the top window 330a to the right graphic 350. The image 370 may be generated by the left camera, and the image 372 may be generated by the right camera.

Turning to the components, a top window 330 (330a or 330b) may have any suitable shape, size, and/or location. In one or more embodiments, a top window 330 (330a or 330b) has a shape, size, and/or location that allows a line of sight (390 or 392, respectively) to pass through to a corresponding graphic (348 or 350, respectively). A top window 330 (330a or 330b) may be formed in any suitable manner. For example, a top window 330 (330a or 330b) may be an indentation formed by the edge of an overhang 324 (324a or 342b, respectively). As another example, a top window 330 (330a or 330b) may be a transparent portion of an overhang 324 (324a or 342b, respectively). In this example, the transparent portion may have a window graphic 332 (332a or 332b, respectively).

In the example, when the right camera is aligned, the line of sight 392 of the right camera passes through the top window 330a to the right graphic 450, but the rest of the wall 306 blocks the line of sight 392 to the left graphic 348. The image 372 taken by the right camera shows the right graphic 350 through the top window 330a. If the top window 330a has a window graphic 332a, the image 372 shows the window graphic 332a relative to the right graphic 350. In certain embodiments, a spatial relationship between the window graphic 332a and the right graphic 350 indicates the alignment of the right camera. In the example, the image 372 shows the right graphic 350 inside of a circle of the window graphic 332a, so the right camera is aligned.

Similarly, when the left camera is aligned, the line of sight 390 of the left camera to passes through the top window 330b to the left graphic 348, but the rest of the wall 306 blocks the line of sight 390 to the right graphic 350. The image 370 taken by the left camera shows the left graphic 348 through the top window 330b. If the top window 330b has a window graphic 332b, the image 370 shows the window graphic 332b relative to the left graphic 348. In certain embodiments, a spatial relationship between the window graphic 332b and the left graphic 348 indicates the alignment of the left camera. In the example, the image 370 shows the left graphic 348 inside of circle of the window graphic 332b, so the left camera is aligned.

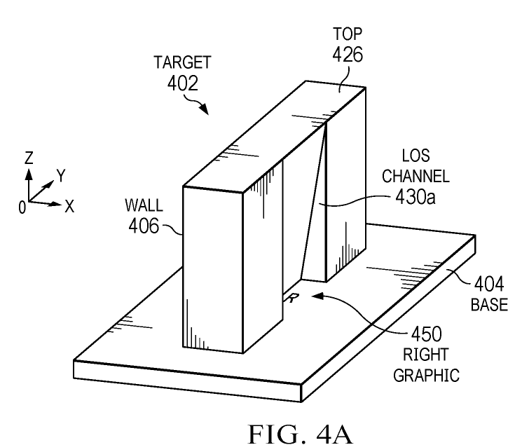

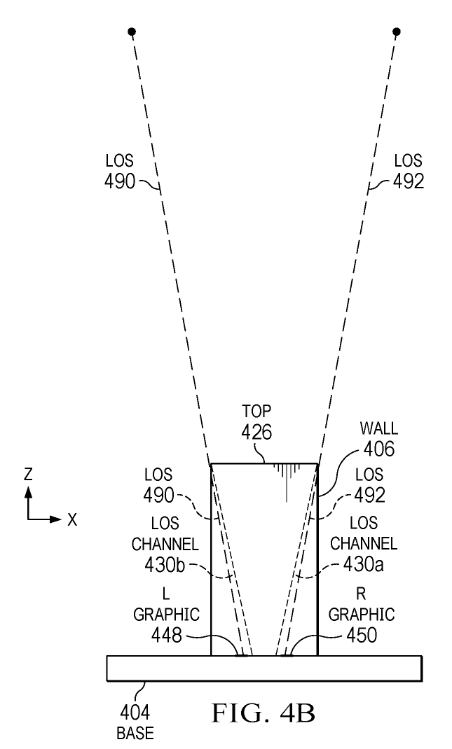

FIGS. 4A and 4B illustrate an example of a target 402 with one or more line of sight (LOS) channels 430 (430a and/or 430b), according to at least one embodiment described in the present disclosure. In the example, the target 402 may be used to test a left camera and a right camera of an imaging system. FIG. 4A shows a perspectival view of the target 402. FIG. 4B shows the target 402 relative to a line of sight (LOS) 490 of the left camera and a line of sight (LOS) 492 of the right camera when the right camera and the left camera are aligned with the target 402.

In the example, the target 402 includes a base 404 and a wall 406. The base has a left graphic 448 and a right graphic 450. The wall 406 has a top 426 and one or more line of sight (LOS) channels 430 (430a and/or 430b). The wall 406 may have any suitable size and/or shape. In one or more embodiments, the wall 406 has a substantially rectangular cuboid shape, where an upper surface of the wall forms the top 426 of the wall 406. The wall 406 may have any suitable height in the z-direction, e.g., 1 to 10 centimeters (cm), such as 1 to 2 cm, 2 to 4 cm, 4 to 6 cm, and/or 6 to 10 cm. The wall 406 may have any suitable length in the y-direction, e.g., 1 to 10 centimeters (cm), such as 1 to 2 cm, 2 to 4 cm, 4 to 6 cm, and/or 6 to 10 cm. The wall 406 may have any suitable thickness in the x-direction, e.g., 1 to 20 millimeters (mm), such as 1 to 5 mm, 5 to 10 mm, 10 to 15 mm, and/or 15 to 20 mm.

In one or more embodiments, a line of sight channel 430 (430a or 430b) has a shape, size, and/or location that allows a line of sight (492 or 490, respectively) of a camera to pass through to a corresponding graphic (450 or 448, respectively) when the camera is aligned. A line of sight channel 430 (430a or 430b) may be formed in any suitable manner. For example, a line of sight channel 430 (430a or 430b) may be an indentation formed by a side of the wall 406. As another example, a line of sight channel 430 (430a or 430b) may be a transparent portion of the wall 406.

In the example, when the right camera is aligned, the line of sight channel 430a allows the line of sight 492 of the right camera to pass through to the right graphic 450, but the rest of the wall 406 blocks the line of sight 492 to the left graphic 448. Accordingly, when aligned, the right camera generates a right image that shows the right graphic 450 but not the left graphic 448. Similarly, when the left camera is aligned, the line of sight channel 430b allows the line of sight 490 of the left camera to pass through to the left graphic 448, but the rest of the wall 406 blocks the line of sight 490 to the right graphic 450. Accordingly, when aligned, the left camera generates a left image that shows the left graphic 448 but not the right graphic 450.

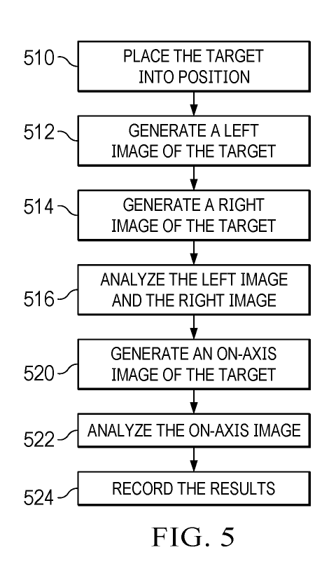

FIG. 5 illustrates an example of a method of using a target to test an imaging system, according to at least one embodiment described in the present disclosure. The blocks of the method may be performed by a system, such as an ophthalmic system, described herein. The ophthalmic system may include a computer system, the imaging system (which has an optical axis), and/or a platform on which the target may be placed. The imaging system may include one or more cameras, such as a left camera, a right camera, and/or an on-axis camera. The target may include one or more graphics, e.g., a distance test graphic, a pilot test graphic, a left graphic, a right graphic, a field-of-view test graphic, a resolution test graphic, and/or a color test graphic.

At block 510, the target is placed into position for testing the ophthalmic system. In one or more embodiments, a user may use a graphic of the target to place the target into position. For example, the user may use the distance test graphic to place the target at the proper distance along the optical axis from the system. As another example, the user may use the pilot test graphic to place the target at the proper location on a plane orthogonal to the optical axis. In one or more embodiments, the computer system may determine if the target is in the correct position. If the target is not in the correct position, the computer system may move the platform to move the target to the correct position.

At block 512, the left camera generates a left image of the target to test the alignment of the left camera. If the left camera is properly aligned, the target allows the line of sight of the left camera to reach the left graphic but blocks the line of sight from reaching the right graphic.

At block 514, the right camera generates a right image of the target to test the alignment of the right camera. If the right camera is properly aligned, the target allows the line of sight of the right camera to reach the right graphic but blocks the line of sight from reaching the left graphic.

At block 516, the computer system analyzes the left image and the right image. If the left image shows the left graphic but not the right graphic, the computer system determines that the left camera is aligned. Otherwise, the computer system may determine that the left camera is not aligned and may provide a notification of the misalignment. Similarly, if the right image shows the right graphic but not the left graphic, the computer system determines that the right camera is aligned. Otherwise, the computer system may determine that the right camera is not aligned and may provide a notification of the misalignment.

The computer system may analyze other graphics of the images. For example, the computer system may analyze the field-of-view test graphic, the resolution test graphic, and/or the color test graphic to test the field-of-view, the resolution, and/or the color response, respectively, of the left camera and/or right camera.

At block 520, the on-axis camera generates an on-axis image of the target.

At block 522, the computer system analyzes the on-axis image. For example, the computer system may analyze the field-of-view test graphic, the resolution test graphic, and/or the color test graphic in the on-axis image to test the field-of-view, the resolution, and/or the color response, respectively, of the on-axis camera.

At block 524, the computer system records the results. For example, the computer system may save the images and/or the results of the analyses.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to” or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

Additionally, relative terms may be used and understood as typically used in the relevant art. By way of example, relative terms such as “approximately,” “substantially,” “about,” “roughly,” or other similar relative terms mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When such relative terms are used to refer to numbers or values that are part of a range, the term can be used to modify both the minimum and maximum numbers or values.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. §112(f).

What is claimed:

1. A target configured to test an imaging system, the target comprising:
a base comprising an upper base surface, a plurality of graphics located on the upper base surface, the plurality of graphics configured to facilitate testing of the imaging system, the imaging system comprising a plurality of cameras, the plurality of cameras comprising a first camera and a second camera, the plurality of graphics comprising a first graphic and a second graphic; and
a wall disposed outwardly from the base, the wall having a top, the top disposed on a side opposite of the base, the wall configured to, when the first camera and the second camera are aligned:
allow the first camera to image the first graphic;
allow the second camera to image the second graphic;
block the first camera from imaging the second graphic; and
block the second camera from imaging the first graphic.

2. The target of claim 1, the top having a top midplane, the wall having a wall midplane, the top midplane substantially orthogonal to the wall midplane.

3. The target of claim 1, the wall having a wall midplane, the base having a base midplane, the wall midplane substantially orthogonal to the base midplane.

4. The target of claim 1, the top comprising an upper top surface, one or more graphics of the plurality of graphics located on the upper top surface.

5. The target of claim 1, the plurality of graphics comprising:
a color test graphic configured to facilitate testing of a color response of at least one camera of the plurality of cameras.

6. The target of claim 1, the plurality of graphics comprising:
a resolution test graphic configured to facilitate testing of a resolution of at least one camera of the plurality of cameras.

7. The target of claim 1, the plurality of graphics comprising:
a field-of-view test graphic configured to facilitate testing of a field-of-view of at least one camera of the plurality of cameras.

8. The target of claim 1, the plurality of graphics comprising:
a pilot test graphic configured to indicate an aligned location of a pilot laser spot.

9. The target of claim 1, the plurality of graphics comprising:
a distance test graphic configured to indicate at least one aligned location of a distance laser spot.

10. The target of claim 1:
the imaging system having an optical axis;
the first camera comprising a first off-axis camera relative to the optical axis; and
the second camera comprising a second off-axis camera relative to the optical axis.

11. The target of claim 1, the top having a top midplane, the top comprising at least one overhang extending along the top midplane away from the wall.

12. The target of claim 11, the at least one overhang comprising a top window, the top window comprising an indentation defined by an edge of the at least one overhang.

13. The target of claim 11, the at least one overhang comprising a top window, the top window comprising a transparent portion of the at least one overhang.

14. The target of claim 11, the top comprising a plurality of overhangs comprising the at least one overhang, the plurality of overhangs comprising:
a first overhang comprising a first top window configured to allow the first camera to image the first graphic; and
a second overhang comprising a second top window configured to allow the second camera to image the second graphic.

15. The target of claim 11, the at least one overhang comprising a top window with a window graphic, the top window configured to:
allow the first camera to image the first graphic and the window graphic, a spatial relationship between the first graphic and the window graphic indicating an alignment of the first camera.

16. The target of claim 1, the wall having a rectangular cuboid shape, an upper surface of the wall forming the top of the wall, the wall comprising a line of sight channel configured to:
allow the first camera to image the first graphic.

17. The target of claim 16, the line of sight channel comprising an indentation of the wall.

18. The target of claim 16, the line of sight channel comprising a transparent portion of the wall.

19. An ophthalmic system, comprising:
an imaging system comprising:
a first camera configured to generate a first image of a target; and
a second camera configured to generate a second image of the target, the target comprising a base and a wall, the wall disposed outwardly from the base, the base comprising a first graphic and a second graphic designed to facilitate testing of the imaging system, the wall configured to, when the first camera and the second camera are aligned, allow the first camera to image the first graphic, allow the second camera to image the second graphic, block the first camera from imaging the second graphic, and block the second camera from imaging the first graphic; and
a computer system configured to:
establish that the first image shows the first graphic but not the second graphic;
establish that the second image shows the second graphic but not the first graphic; and
determine that the first camera and the second camera are aligned.

20. The ophthalmic system of claim 19:
further comprising a laser system configured to direct a pilot beam towards the target to yield a pilot beam spot on the target, the pilot beam configured to indicate a direction of a laser beam provided by the laser system, the target comprising a pilot test graphic indicating an alignment of the pilot beam;
the imaging system configured to:
generate a pilot beam image of the pilot beam spot on the target; and
the computer system configured to:
determine the alignment of the pilot beam relative to the target according to a location of the pilot beam spot relative to the pilot test graphic.

21. The ophthalmic system of claim 19:
further comprising an alignment light system configured to direct a plurality of off-axis light beams towards the target to yield a plurality of off-axis light spots on the target, the plurality of off-axis light beams indicating an aligned distance between the ophthalmic system and the target, the target comprising a distance test graphic corresponding to the plurality of off-axis light beams;
the imaging system configured to:
generate a distance image of the plurality of off-axis light spots on the target; and
the computer system configured to:
determine whether a distance between the ophthalmic system and the target corresponds to the aligned distance according to a location of the plurality of off-axis light spots relative to the distance test graphic.

22. The ophthalmic system of claim 19:
further comprising a platform configured to support the target; and
the computer system configured to:
determine a misalignment of the target relative to the ophthalmic system; and
instruct the platform to move to compensate for the misalignment.
